(19) United States
(12) Patent Application Publication
(10) Pub. No.: US 2019/0269381 A1
(43) Pub. Date: Sep. 5, 2019

(54) ULTRASONIC CROSS-SECTIONAL IMAGE MEASUREMENT APPARATUS

(71) Applicant: UNEX CORPORATION, Nagoya-shi, Aichi (JP)

(72) Inventors: Hiroshi MASUDA, Nagoya-shi (JP); Chikao HARADA, Nagoya-shi (JP); Hiromasa TSUKAHARA, Nagoya-shi (JP); Hidenori SUZUKI, Nagoya-shi (JP)

(73) Assignee: UNEX CORPORATION, Nagoya-shi, Aichi (JP)

(21) Appl. No.: 16/303,584

(22) PCT Filed: May 22, 2017
(86) PCT No.: PCT/JP2017/019092

§ 371 Date: Nov. 20, 2018

(30) Foreign Application Priority Data
May 27, 2016 (JP) 2016-106841

(51) Int. Cl. A61B 8/08; A61B 5/02

(52) U.S. Cl. CPC A61B 8/0891; A61B 5/02007

(57) ABSTRACT

An ultrasonic cross-sectional image measuring device has: a living body compressing device including an annular compression band wrapped around a portion of the living body, an ultrasonic transmission plate material allowing transmission of ultrasonic waves, disposed on a portion of the compression band, and brought into close contact with the portion of the living body, and an actuator adjusting a tension of the compression band to change a compression pressure of the ultrasonic transmission plate material to the living body; a container including an opening closed by the ultrasonic transmission plate material and filled with a liquid; an ultrasonic probe housed in the container and transmitting and receiving ultrasonic waves through the ultrasonic transmission plate material to and from the portion of the living body; and a control device generating an ultrasonic cross-sectional image based on an ultrasonic signal received by the ultrasonic probe.

### TECHNICAL FIELD

The present invention relates to an ultrasonic image measuring device having a living body compressing device capable of measuring a cross-sectional image of a tubular organ in a living body corresponding to a change in compression pressure from the living body compressing device.

### BACKGROUND ART

Regarding organs in a living body, studies are conducted on the possibility of making determination, diagnosis, etc. of the organs by measuring an amount of deformation of the organs under a predetermined compression pressure from the outside in an ultrasonic cross-sectional image, and it is desired in the fields of biological diagnosis etc. to measure a cross-sectional shape of an organ in a living body or a tubular organ such as an artery and a vein in a living body from an ultrasonic image while changing a degree of compression to the living body by the living body compressing device.

In an ultrasonic cross-sectional image measuring device disclosed in Patent Document 1, an ultrasonic cross-sectional image of an organ in a living body can be acquired by using an ultrasonic array in which multiple ultrasonic transducers (ultrasonic oscillators) made up of piezoelectric ceramics etc. are arranged in a line and which is housed in a liquid-tight container and by linearly reciprocating the ultrasonic array in a short axis direction with a bottom surface of the container brought into close contact with the living body.

According to this device, a possibility of presence of a tumor in the living body can be found out from the ultrasonic cross-sectional image in the living body in a state where the device is in close contact with the living body (breast).

### PRIOR ART DOCUMENT

### Patent Document

Patent Document 1: Japanese Laid-Open Patent Publication No. 2008-80093

### SUMMARY OF THE INVENTION

### Problem to be Solved by the Invention

However, the ultrasonic image measuring device described in Patent Document 1 does not include a living body compressing device applying a compression pressure to a living body, which makes it difficult to accurately measure a cross-sectional image of an organ in a living body corresponding to a change in compression pressure to the living body at a part compressed by the living body compressing device.

The present invention was conceived in view of the situations and it is therefore an object of the present invention to provide an ultrasonic cross-sectional image measuring device having a living body compressing device capable of measuring a cross-sectional image of an organ in a living body corresponding to a change in compression pressure to the living body at a part compressed by the living body compressing device.

As a result of various studies conducted in view of the situations, the present inventors found that when an ultrasonic transmission plate material which transmits ultrasonic waves is disposed on a portion of an annular compression band wound around a portion of a living body to tighten the portion of the living body, and ultrasonic waves are transmitted into the living body from an ultrasonic probe through the ultrasonic transmission plate material and received, a shape of an ultrasonic cross-sectional image of an organ in a living body corresponding to a change in compression pressure can be acquired. The present invention was conceived based on such findings.

### Solution to Problem

To achieve the above object, a first aspect of the present invention provides an ultrasonic cross-sectional image measuring device (a) measuring an ultrasonic cross-sectional image in a living body corresponding to a change in compression pressure to the living body, comprising: (b) a living body compressing device including an annular compression band wrapped around a portion of the living body for tightening the portion of the living body, an ultrasonic transmission plate material allowing transmission of ultrasonic waves, disposed on a portion of the compression band, and brought into close contact with the portion of the living body, and an actuator adjusting a tension of the compression band to change a compression pressure of the ultrasonic transmission plate material to the living body; (c) a container including an opening closed by the ultrasonic transmission plate material and filled with a liquid; (d) an ultrasonic probe housed in the container and transmitting and receiving ultrasonic waves through the ultrasonic transmission plate material to and from the portion of the living body; and (e) a control device generating an ultrasonic cross-sectional image based on an ultrasonic signal received by the ultrasonic probe.

A second aspect of the present invention provides the ultrasonic cross-sectional image measuring device recited in the first aspect of the invention, wherein the control device changes the compression pressure applied to the portion of the living body by the living body compressing device based on the ultrasonic cross-sectional image.

A third aspect of the present invention provides the ultrasonic cross-sectional image measuring device recited in the first or second aspect of the invention, wherein the control device controls the compression pressure applied to the portion of the living body by the living body compressing device so as to maintain a predetermined number of pulses in which an artery in the living body is put into a collapsed state in a portion of each pulse wave period of the living body based on the ultrasonic cross-sectional image when vascular dilation of the living body is measured.

A fourth aspect of the present invention provides the ultrasonic cross-sectional image measuring device recited in any one of the first to third aspects of the invention, wherein after controlling the compression pressure applied to the portion of the living body by the living body compressing device so as to maintain a predetermined number of pulses in which an artery in the living body is flatly compressed in each pulse wave period of the living body based on the ultrasonic cross-sectional image so that a shear stress is applied to the artery in the living body, the control device releases the compression applied by the living body compressing device and calculates a diameter expansion ratio of the artery based on the ultrasonic cross-section image.

A fifth aspect of the present invention provides the ultrasonic cross-sectional image measuring device recited in any one of the first to fourth aspects of the invention, wherein the control device calculates and outputs an index indicative of a stiffness of a blood vessel in the living body based on a ratio between a change in shape of the blood vessel in the living body obtained from the ultrasonic cross-sectional image and a change in the compression pressure applied by the compressing device.

A sixth aspect of the present invention provides the ultrasonic cross-sectional image measuring device recited in any one of the first to fifth aspects of the invention, wherein before puncturing a blood vessel in the living body, the control device determines whether the blood vessel is a vein based on whether the blood vessel is flatly compressed by increasing the compression pressure applied by the compressing device.

### Advantageous Effects of Invention

The ultrasonic cross-sectional image measuring device recited in the first aspect of the invention measures (a) the ultrasonic cross-sectional image in the living body corresponding to the change in compression pressure to the living body and comprises: (b) the living body compressing device having the annular compression band wrapped around a portion of the living body for tightening the portion of the living body, the ultrasonic transmission plate material that is disposed on a portion of the compression band and that can be brought into close contact with the portion of the living body, and the actuator capable of adjusting the tension of the compression band to change the compression pressure of the ultrasonic transmission plate material to the portion of the living body; (c) the container having the opening closed by the ultrasonic transmission plate material and filled with the liquid; (d) the ultrasonic probe housed in the container and transmitting and receiving ultrasonic waves through the ultrasonic transmission plate material to and from the portion of the living body; (e) and the control device generating the ultrasonic cross-sectional image based on the ultrasonic signal received by the ultrasonic probe, and therefore, the cross-sectional image in the living body compressed by the living body compressing device is accurately obtained. Specifically, since the portion of the living body is fixed by the annular compression band, the influence of body motion is avoided, and the part of the living body compressed by the ultrasonic transmission plate material of the living body compressing device coincides with the position of the cross-sectional image in the living body obtained through the ultrasonic transmission plate material by the ultrasonic probe, so that the shape of the cross-sectional image in the living body with respect to the compression pressure by the living body compressing device can accurately be obtained.

In the ultrasonic cross-sectional image measuring device recited in the second aspect of the invention, the control device changes the compression pressure applied to a portion of the living body by the living body compressing device based on the ultrasonic cross-sectional image, so that the compression pressure can be changed such that the blood vessel in the living body in the ultrasonic cross-sectional image has a desired shape. For example, the control device determines a state in which the blood vessel is collapsed into a flatly pressed state, i.e., a flat shape, based on the cross-sectional shape of the blood vessel, and can change the compression pressure applied to the portion of the living body by the living body compressing device such that the flatly pressed state is achieved in a portion or whole of pulse period of one beat.

In the ultrasonic cross-sectional image measuring device recited in the third aspect of the invention, the control device controls the compression pressure applied to the portion of the living body by the living body compressing device so as to maintain the predetermined number of pulses in which the artery in the living body is put into the collapsed state in a portion of each pulse wave period of the living body based on the ultrasonic cross-sectional image when the vascular dilation of the living body is measured. As a result, a turbulent flow is repeatedly generated in synchronization with pulses in the artery of the living body, so that a shear stress is efficiently applied to the endothelium of the artery of the living body. For example, as compared to a conventional FMD (flow-mediated dilation) measurement in which the shear stress is applied by releasing the artery after five minutes of ischemia, the shear stress is applied in a short time. Therefore, the FMD measurement can be performed in a short time.

In the ultrasonic cross-sectional image measuring device recited in the fourth aspect of the invention, after controlling the compression pressure applied to the portion of the living body by the living body compressing device so as to maintain the predetermined number of pulses in which the artery of the living body is collapsed in each pulse wave period of the living body based on the ultrasonic cross-sectional image so that the shear stress is applied to the artery of the living body, the control device releases the compression applied by the living body compressing device and calculates a diameter expansion ratio of the artery of the living body based on the ultrasonic cross-sectional image, and therefore, the FMD measurement is performed in a short time.

In the ultrasonic cross-sectional image measuring device recited in the fifth aspect of the invention, the control device calculates and outputs an index indicative of the stiffness of the arterial vessel in the living body based on the ratio between a change in the shape of the arterial vessel in the living body obtained from the ultrasonic cross-sectional image and a change in the compression pressure applied by the living body compressing device, and therefore, diagnosis can be made based on the stiffness of the arterial vessel. For example, diagnosis can more accurately be made for arteriosclerosis by combining with the diameter expansion ratio of the artery after applying the shear stress to the artery in the living body.

In the ultrasonic cross-sectional image measuring device recited in the sixth aspect of the invention, the control device determines whether the blood vessel is a vein based on whether the blood vessel is collapsed due to an increase in the compression pressure applied by the living body compressing device before a puncturing operation to the blood vessel in the living body. This eliminates misidentification of the blood vessel at the time of the puncturing operation, and positions of a needle and a vein are confirmed from the ultrasonic cross-sectional image during the puncturing operation, so that the operation of puncturing the vein with the needle becomes more reliable and easier.

### BRIEF DESCRIPTION OF THE DRAWINGS

FIG. 1 is a perspective view for explaining an arterial vessel evaluating device according to an embodiment of the present invention.

FIG. 2 is a perspective view for schematically explaining a posture of an ultrasonic probe relative to a blood vessel to be measured by the arterial vessel evaluating device of FIG. 1.

FIG. 3 is an enlarged view schematically showing a multilayer film configuration of an arterial vessel that is a measurement object of the arterial vessel evaluating device of FIG. 1.

FIG. 4 is a view showing a configuration of a living body compressing device included in the arterial vessel evaluating device of FIG. 1 with a container housing the device partially cut away, and includes a functional block diagram for explaining a main portion of a function of an electronic control device.

FIG. 5 is a functional block diagram for explaining details of a control function of a vascular state evaluating portion of the electronic control device of FIG. 4.

FIG. 6 is a time chart exemplarily showing a change in a vascular lumen diameter in an FMD evaluation operation of an arterial vessel performed in the arterial vessel evaluating device of FIG. 1.

FIG. 7 is a diagram for explaining an operation of changing a compression pressure for applying a shear stress to an arterial vessel endothelium during a shear stress application period of FIG. 6.

FIG. 8 is a diagram for explaining another operation of changing a compression pressure for applying a shear stress to the arterial vessel endothelium during a shear stress application period of FIG. 6.

FIG. 9 is a diagram for explaining still another operation of changing a compression pressure for applying a shear stress to the arterial vessel endothelium during a shear stress application period of FIG. 6.

FIG. 10 is a flowchart for explaining an artery determination routine operation showing an artery determination operation of the vascular state evaluating portion of FIG. 4.

FIG. 11 is a flowchart for explaining an FMD measurement routine operation showing an FMD measurement operation of the vascular state evaluating portion of FIG. 4.

FIG. 12 is a flowchart for explaining an arterial stiffness measurement routine operation showing an arterial stiffness measurement operation of the vascular state evaluating portion of FIG. 4.

### MODES FOR CARRYING OUT THE INVENTION

An embodiment of the present invention will now be described in detail below with reference to the drawings.

### Embodiment

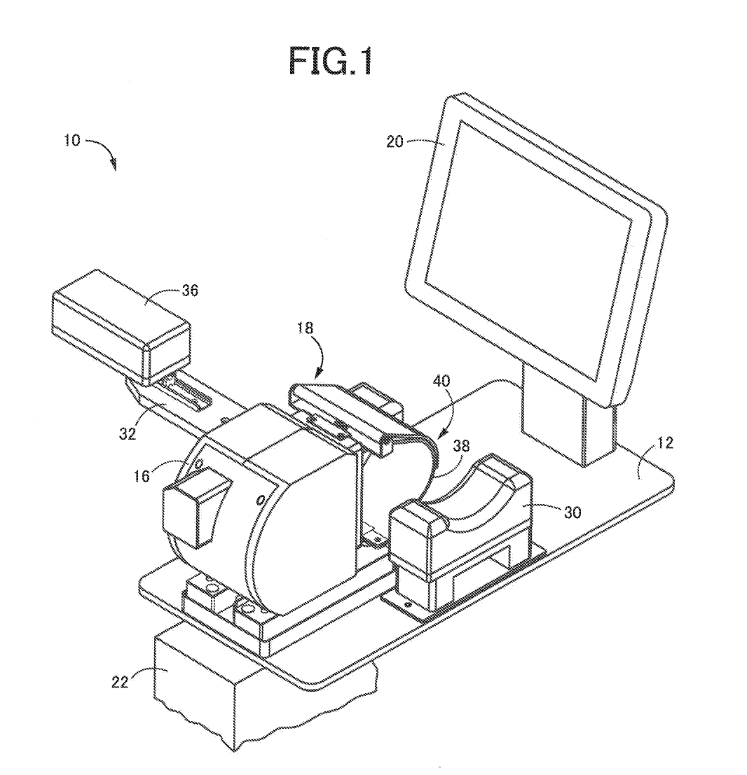

FIG. 1 shows an arterial vessel evaluating device 10 which also functions as an ultrasonic cross-sectional image measuring device having a living body compressing device. The arterial vessel evaluating device 10 includes a closed container 16 fixed on a base 12 and housing an ultrasonic probe 14, a living body compressing device 18 disposed on the closed container 16, a display device 20 fixed on the base 12, and an electronic control device 22 arranged under the base 12.

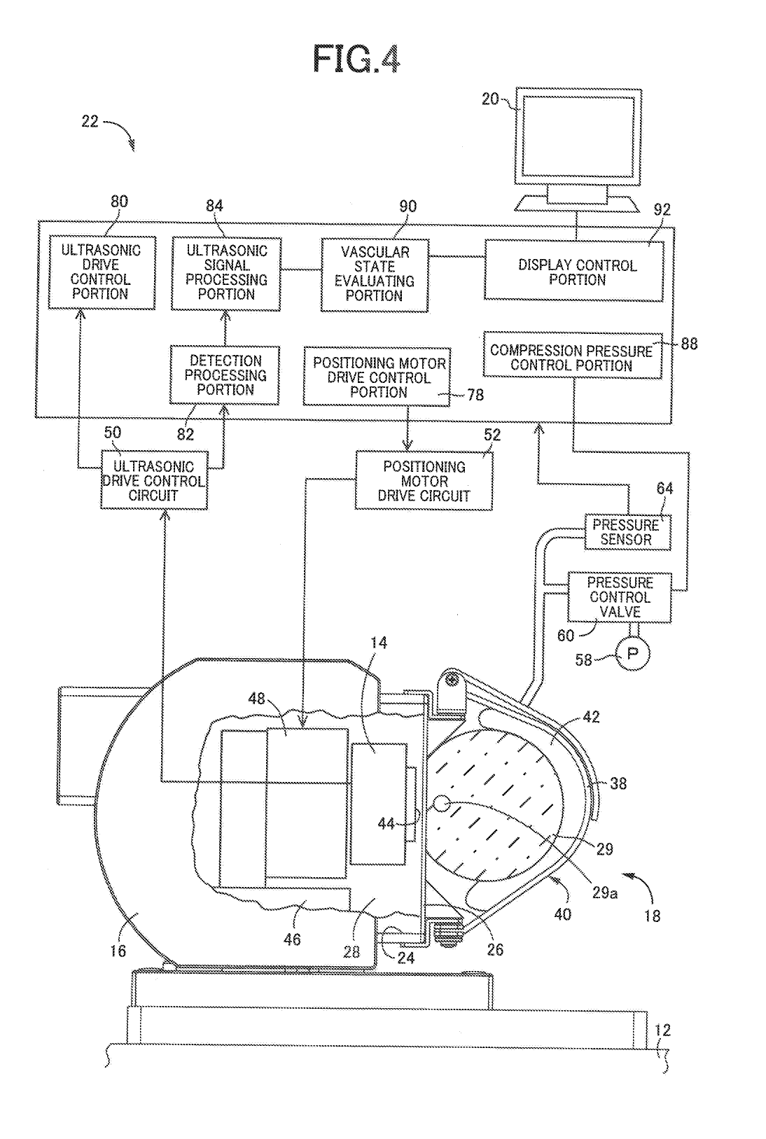

As shown in detail in FIG. 4, the closed container 16 has a laterally-opened opening 24, and the opening 24 is liquid-tightly closed by an ultrasonic transmission plate material 26 made of a material that has an acoustic impedance similar to a living body and a high ultrasonic transmission efficiency, for example, an organic material such as a vinyl acetate based material, that is, the ultrasonic transmission plate material 26 transmits ultrasonic waves. As a result, the inside of the closed container 16 is filled with a liquid ultrasonic medium, for example, an oil 28, having an acoustic impedance similar to a living body and a small propagation loss.

Returning to FIG. 1, the living body compressing device 18 includes an upper arm rest 30 fixed on the base 12 for placing a right upper arm 29 of a living body, a palm rest 36 fixed on a bracket 32 horizontally projected from the base 12 for placing a right palm of the living body, a compression band 40 made up of a flexible belt 38 such that both ends of the flexible belt 38 are respectively attached to an upper opening edge and a lower opening edge of the opening 24 of the closed container 16, and an inflatable bag 42 mounted on the inside of the compression band 40 and inflated to increase the tension of the compression band 40. The ultrasonic transmission plate material 26 substantially constitutes a portion of the compression band 40. In the living body compressing device 18, when the inflatable bag 42 is inflated by supplying compressed air with the right upper arm 29 of the living body wrapped with the compression band 40, the tension of the compression band 40 is increased, and at the same time, the right upper arm 29 of the living body is pressed against the ultrasonic transmission plate material 26 so that the right upper arm 29 of the living body is compressed by the ultrasonic transmission plate material 26.

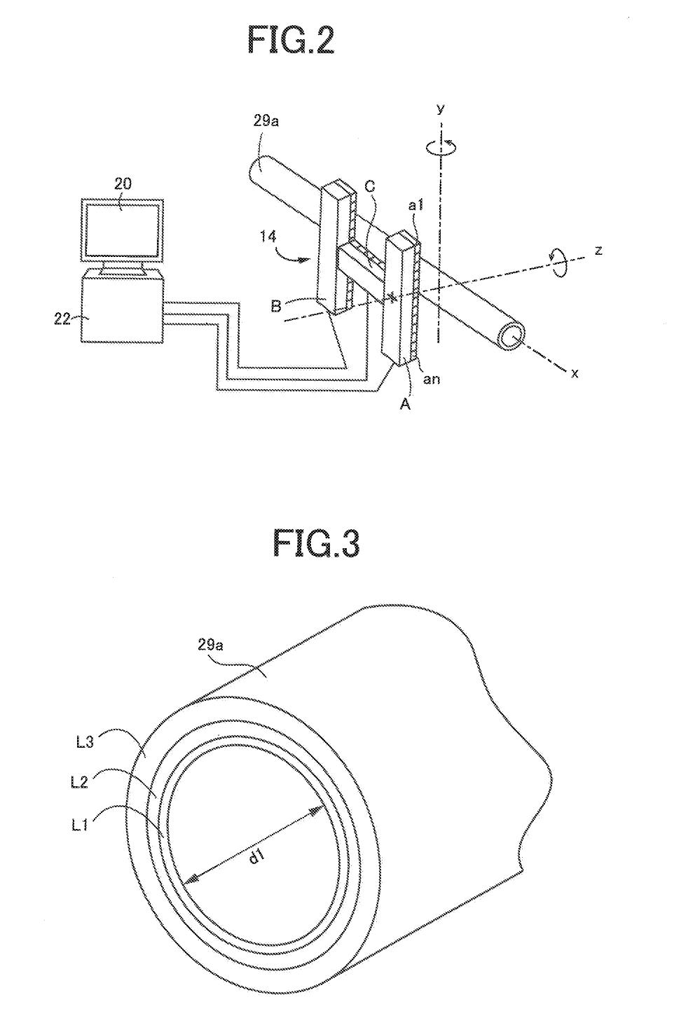

The ultrasonic probe 14 functions as a sensor for detecting biological information related to an arterial vessel 29a in the right upper arm 29 of the living body, i.e., a vascular parameter, and is an H-shaped ultrasonic probe having, as shown in FIG. 2, a pair of a first short-axis ultrasonic array probe A and a second short-axis ultrasonic array probe B parallel to each other as well as a long-axis ultrasonic array probe C forming an elongated shape in a direction orthogonal to a longitudinal direction of the ultrasonic array probes A and B and coupling longitudinal-direction central portions of the ultrasonic array probes A and B, on one flat surface, i.e., a flat probe surface 44. As shown in FIG. 4, the ultrasonic probe 14 is fixed to a multi-axis positioning device 48 fixed to a base member 46. The first short-axis ultrasonic array probe A, the second short-axis ultrasonic array probe B, and the long-axis ultrasonic array probe C are, for example, as shown in FIG. 2 described later, each formed into an elongated shape by linearly arranging a multiplicity of ultrasonic transducers (ultrasonic oscillators) al to an made of piezoelectric ceramics.

FIG. 2 is a perspective view showing the first short-axis ultrasonic array probe A and the second short-axis ultrasonic array probe B disposed parallel to each other in the ultrasonic probe 14 as well as the long-axis ultrasonic array probe C disposed between the longitudinal-direction central portions of the first short-axis ultrasonic array probe A and the second short-axis ultrasonic array probe B orthogonally thereto. In the multi-axis positioning device 48, when a y axis is defined such that the y axis is parallel to the longitudinal direction of the first short-axis ultrasonic array probe A, and passes through the arterial vessel 29a which locates in an ultrasonic beam radiation direction of the first short-axis ultrasonic array probe A or the vicinity thereof and an x axis is a direction parallel to the longitudinal direction of the long-axis ultrasonic array probe C and orthogonal to the y axis while a z axis is a direction passing through an intersection of the longitudinal direction of the first short-axis ultrasonic array probe A and the longitudinal direction of the long-axis ultrasonic array probe C and orthogonal to the x-axis direction and the y-axis direction, the ultrasonic probe 14 can be translated in the y-axis direction and pivoted around each of the y and z axes by the multi-axis positioning device 48.

FIG. 3 is an enlarged view schematically showing a multilayer film configuration of the arterial vessel 29a that is a measurement object of the arterial vessel evaluating device 10. The arterial vessel 29a shown in FIG. 3 has a three-layer structure of an intima (endothelium) L1, a media L2, and an adventitia L3. Reflection of ultrasonic waves generally occurs at a portion having a different acoustic impedance, and therefore, in measurement of a state of the arterial vessel 29a using ultrasonic waves, actually, an interface between a blood in a vascular lumen and the intima L1, and an interface between the media L2 and the adventitia L3 are displayed in white and a tissue is displayed as a black-and-white pattern.

The electronic control device 22 is a so-called microcomputer having a CPU processing an input signal in accordance with a program stored in advance in a ROM while utilizing a temporary storage function of a RAM. The electronic control device 22 includes an ultrasonic drive control circuit 50 and a positioning motor drive circuit 52. In measurement of a vascular state by the arterial vessel evaluating device 10, when a drive signal is supplied from the ultrasonic drive control circuit 50 by the electronic control device 22, beam-like ultrasonic waves are sequentially radiated from the first short-axis ultrasonic array probe A, the second short-axis ultrasonic array probe B, and the long-axis ultrasonic array probe C of the ultrasonic probe 14 by well-known beam forming drive. Reflection signals of the ultrasonic waves are detected by the first short-axis ultrasonic array probe A, the second short-axis ultrasonic array probe B, and the long-axis ultrasonic array probe C and input to the electronic control device 22. The reflected wave signals input to the electronic control device 22 are detected by a detection processing portion 82 and processed by an ultrasonic signal processing portion 84 as information usable for image synthesis. As a result, an ultrasonic two-dimensional cross-sectional image under skin is generated and displayed on the display device 20 functioning as a monitor screen display device or an image display device.

The multi-axis positioning device 48 includes a y-axis pivoting mechanism for positioning a pivotal position around the y axis of the ultrasonic probe 14 by a y-axis pivoting motor, a y-axis translating mechanism for positioning the ultrasonic probe 14 in the z-axis direction by a y-axis translating motor, and a z-axis pivoting mechanism for positioning a pivotal position around the z axis of the ultrasonic probe 14 by a z-axis pivoting motor. The positioning motor drive circuit 52 controls the y-axis pivoting motor, the y-axis translating motor, and the z-axis pivoting motor in accordance with a command from the electronic control device 22.

As shown in FIG. 4, the electronic control device 22 includes a positioning motor drive control portion 78, an ultrasonic drive control portion 80, the detection processing portion 82, the ultrasonic signal processing portion 84, a compression pressure control portion 88, a vascular state evaluating portion 90, and a display control portion 92. These control functions are functionally included in the electronic control device 22, but some or all of these control functions may be configured in corresponding electronic control device separated from the electronic control device 22 where electronic control devices communicate each other to provide controls described in detail below.

The electronic control device 22 extracts a vascular cross-sectional image from an ultrasonic cross-sectional image of the blood vessel 29a based on the reflection signals of the ultrasonic waves output from the ultrasonic probe 14 to the arterial vessel 29a, generates an ultrasonic short-axis image showing a cross section orthogonal to the longitudinal direction from the vascular cross-sectional image, measures an inner diameter, an intima thickness, plaque, etc. of the arterial vessel 29a from the ultrasonic short-axis image, and further makes evaluation on FMD (flow-mediated dilation). At the time of the evaluation on FMD, the display device 20 displays a change rate of a maximum diameter d_{MAX} of the intima after application of shear stress to a diameter da of the intima L1 of the arterial vessel 29a at rest, i.e., a dilation rate R of a lumen diameter in time series. At the time of evaluation on FMD, generation of an ultrasonic image of the arterial vessel 29a, etc., the ultrasonic probe 12 repeatedly scans the skin on the arterial vessel 29a to be measured.

In the measurement of the vascular state of the artery 20 by the electronic control device 22, the ultrasonic probe 14 emits ultrasonic wave signals through the ultrasonic transmission plate material 26 and the skin of the upper arm 29 of the living body to the arterial vessel 29a located directly under the skin and receives reflected waves thereof. In this state, based on a position of a first short-axis cross-sectional image of the arterial vessel 29a generated by the ultrasonic signal processing portion 84 from the ultrasonic reflection signal received by the first short-axis ultrasonic array probe A, a position of a second short-axis cross-sectional image of the arterial vessel 29a generated by the ultrasonic signal processing portion 84 from the ultrasonic reflection signal received by the second short-axis ultrasonic array probe B, and a position of a long-axis cross-sectional image of the arterial vessel 29a generated by the ultrasonic signal processing portion 84 from the ultrasonic reflection signal received by the long-axis ultrasonic array probe C, the positioning motor drive control portion 78 automatically positions the ultrasonic probe 14 such that the arterial vessel 29a is located under the longitudinal-direction central portions of the first short-axis ultrasonic array probe A and the second short-axis ultrasonic array probe B while the long-axis ultrasonic array probe C and the arterial vessel 29a are parallel to each other.

The ultrasonic signal processing portion 84 uses propagation speed differences between the arterial vessel 29a and other tissues to perform a time difference process etc. between ultrasonic reflection signals reflected from boundaries therebetween to repeatedly generate image data made up of the first short-axis cross-sectional image that is an ultrasonic two-dimensional image directly under the first short-axis ultrasonic array probe A, the second short-axis cross-sectional image that is an ultrasonic two-dimensional image directly under the second short-axis ultrasonic array probe B, and the long-axis cross-sectional image that is an ultrasonic two-dimensional image directly under the long-axis ultrasonic array probe C in predetermined cycles and sequentially stores the image data.

As shown in FIG. 1, the inflatable bag 42 is inflated to increase the tension of the compression band 40, by controlling an air pump 58, a pressure control valve 60, etc. by the compression pressure control portion 88 included in the electronic control device 22. For example, in accordance with a command from the electronic control device 22, a source pressure from the air pump 58 is controlled by the pressure control valve 60 and supplied to the inflatable bag 42 of the compression band 40 wound around the upper arm 29. Specifically, as the pressure in the inflatable bag 42 is increased, the arterial vessel 29a in the upper arm 29 is compressed. In this embodiment, a portion of the compression band 40 is made up of the ultrasonic transmission plate material 26, and the ultrasonic probe 14 transmits and receives the ultrasonic signals through the ultrasonic transmission plate material 26 to and from a compressed part of the arterial vessel 29a in the upper arm 29, so that a cross-sectional image of the part to be compressed of the arterial vessel 29a is obtained.

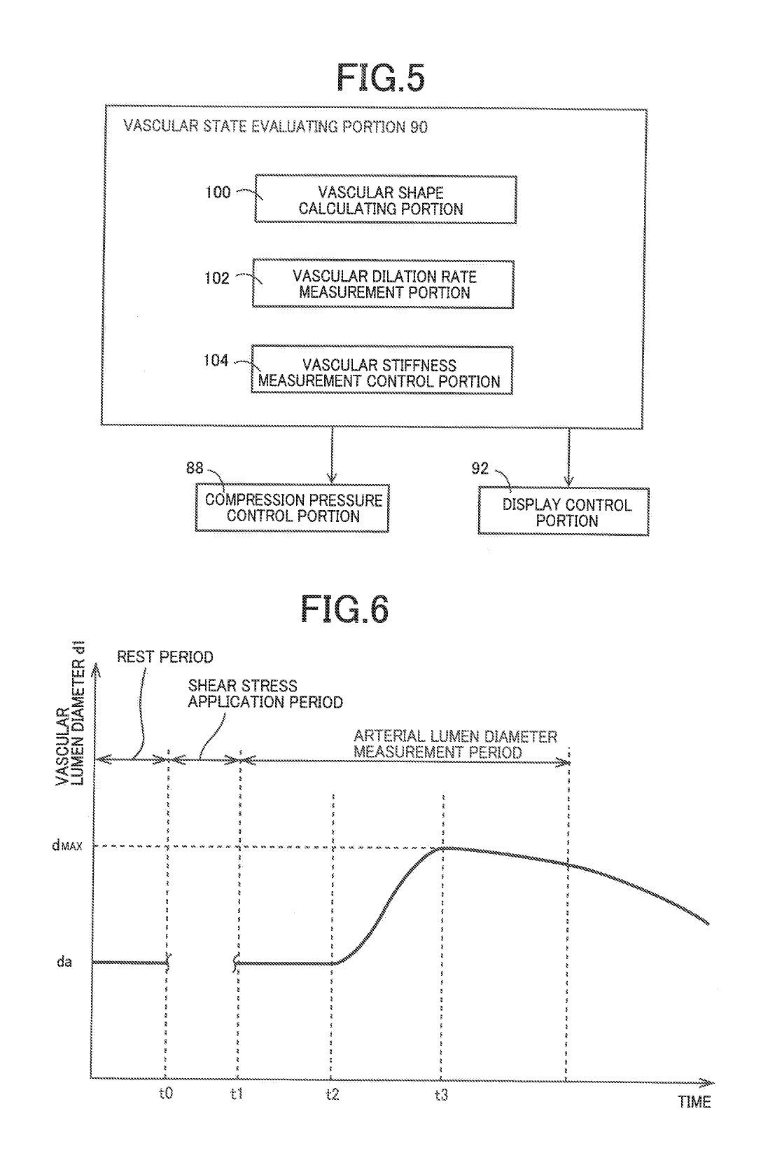

As shown in FIG. 5, the vascular state evaluating portion 90 includes a vascular shape calculating portion 100, a vascular dilation rate measurement control portion 102, and a vascular stiffness measurement control portion 104. From the cross-sectional image of the arterial vessel 29a generated as described above, the vascular shape calculating portion 100 calculates an outer diameter, a wall pressure, an endothelium diameter (lumen diameter) d1 that is the diameter of the endothelium L1, etc. of the arterial vessel 29a.

When the upper arm 29 is compressed by the compression pressure control portion 88 at a pressure higher than a venous pressure and lower than a diastolic blood pressure value Pd, the vascular shape calculating portion 100 executes a process of determining and identifying as the arterial vessel 29a in the ultrasonic cross-sectional images a tubular organ not collapsed in an image showing multiple tubular organs present in the ultrasonic cross-sectional images. The arterial vessel 29a identified in this way is measured as described later in terms of the diameter of the arterial vessel 29a, the endothelium diameter (lumen diameter) d1 that is the diameter of the endothelium L1 of the arterial vessel 29a, a dilation rate (change rate) R (%) of the vascular lumen diameter d1 of the arterial vessel 29a indicative of FMD (flow-mediated dilation) after ischemic reactive hyperemia of the arterial vessel 29a, a systolic blood pressure value Ps and the diastolic blood pressure value Pd of the living body, a stiffness parameter β indicative of the stiffness of the arterial vessel 29a, etc. Such an artery identification image process is also useful for puncture.

The vascular dilation rate measurement control portion 102 successively calculates the endothelium diameter (lumen diameter) d1 temporarily expanded due to flow-mediated dilation after applying a shear stress utilizing a blood flow to the endothelium L1 of the arterial vessel 29a by the compression band 40 wrapped around the upper arm 29 to calculate the dilation rate (change rate) R (%) [=100*(d_{MAX}−da)/da] of the vascular lumen diameter d1 indicative of FMD (flow-mediated dilation) after application of the shear stress. In this equation, “da” denotes a vascular lumen diameter at rest (base diameter, resting diameter). The vascular state evaluating portion 90 also functions as a measuring device for the dilation rate (change rate) R of the vascular lumen diameter d1 indicative of the FMD (flow-mediated dilation) after application of the shear stress.

In the measurement of the dilation rate (change rate) R (%) of the arterial vessel 29a by the vascular dilation rate measurement control portion 102, the measurement part, for example, the upper arm 29, of the living body 14 is compressed by the compression band 40 of the living body compressing device 18 so that the shear stress utilizing a blood flow is applied to the endothelium L1 of the arterial vessel 29a, which causes production of nitric oxide (NO) from the endothelium due to an increase in the shear stress to the endothelium L1 of a vascular wall, and the endothelial function of the arterial vessel 29a is determined by examining the endothelium diameter (lumen diameter) d1 indicating a smooth muscle relaxation status dependent on nitric oxide.

FIG. 6 is a time chart exemplarily showing a change in the vascular lumen diameter d1 after release of ischemia (avascularization) in the FMD evaluation of the arterial vessel 29a by the vascular dilation rate measurement control portion 102. FIG. 6 includes a rest period before time t0, a shear stress application period from time t0 to time t1, and a measurement period of the flow-mediated dilation after application of the shear stress after time t1 and shows that the vascular lumen diameter d1 starts expanding from time t2 and that the vascular lumen diameter d1 reaches the maximum value d_{MAX} at time t3. Therefore, the dilation rate R of the vascular lumen diameter d1 calculated by the electronic control device 22 is maximized at time t3.

To generate vasodilation at the time of FMD evaluation of the brachial artery 29a as described above, conventionally, the brachial artery 29a is compressed (to cause ischemia) at a position upstream or downstream of a part measured with the ultrasonic cross-sectional images, by using a cuff etc. for a predetermined time, for example, five minutes, at a pressure higher than the systolic blood pressure value by about 50 mmHg, for example, and is then quickly released to the atmospheric pressure in about 0.6 seconds, for example, to start a blood flow, which has been zero, and a shear stress is thereby applied to the brachial artery 29a. In such a conventional method, a person to be measured is forced to suffer from compression for five minutes at a pressure considerably higher than the systolic blood pressure value. However, the vascular dilation rate measurement control portion 102 of this embodiment regulates and maintains a predetermined compression pressure on the brachial artery 29a using the inflatable bag 42 for a predetermined time T1 or during a predetermined number of pulses such that, in the ultrasonic cross-sectional image of the brachial artery 29a, the brachial artery 29a is observed or determined as being in a collapsed state, for example, a closed state in which a cross section of the brachial artery 29a is closed (e.g., a flatly-pressed state of being flatly compressed and closed), or in a state in which the cross section of the brachial artery 29a is locally narrowed although the cross section is not closed, during a portion of each pulse wave period, for example, around the timing of the diastolic blood pressure Pd, from the ultrasonic cross-sectional image. The brachial artery 29a is thereby opened and closed for each pulse and subjected to the shear stress repeatedly, so that the shear stress is applied at a lower pressure and in a shorter period than the conventional method.

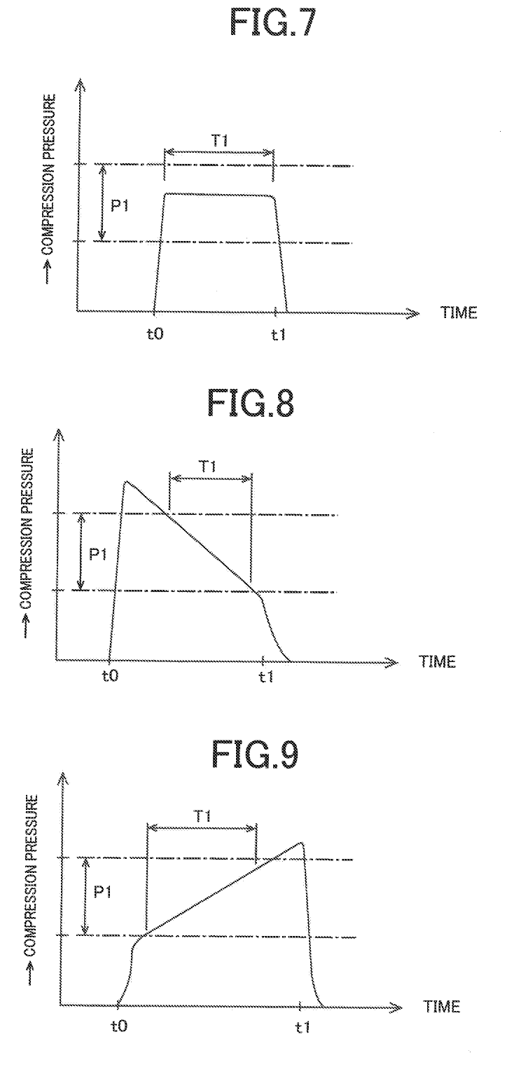

The predetermined compression pressure should be referred to as a shear stress application pressure of efficiently applying a shear stress to the endothelium L1 through the repeated occurrence of turbulence of blood due to the opening and closing of the arterial vessel 29a for each pulse and is set within a pressure range P1 lower than the systolic blood pressure value and higher than the diastolic blood pressure value such that the arterial vessel 29a is put into a collapsed state at a portion of each pulse wave period, for example, near the timing of the diastolic blood pressure Pd. The predetermined time T1 or the predetermined number of pulses is set to a value necessary and sufficient for generating a vascular dilation at the time of FMD evaluation of the arterial vessel 29a, for example, based on an experimental value. The predetermined time T1 or the predetermined number of pulses is set to, for example, several to several tens of seconds, preferably 10 to less than 20 seconds, or several to several tens of beats, preferably 10 to less than 20 beats. The predetermined compression pressure may be controlled to be maintained at a constant value set within the predetermined pressure range P1 for the predetermined time t1 as shown in FIG. 7, for example, or may be controlled to pass through the predetermined pressure range P1 in the predetermined time T1 in an increasing process or a decreasing process at about 5 to 6 mmHg/sec, for example, as shown in FIG. 8 or 9, for example. In short, to generate the vascular dilation at the time of FMD evaluation of the arterial vessel 29a, the compression by the living body compressing device 18 may be controlled within the predetermined pressure range P1 to achieve pulsation having a section in which the arterial vessel 29a is collapsed at a portion of each pulse wave period in the predetermined time T1.

The compression pressure control portion 88 detects the compression pressure according to a signal from a pressure sensor 64 detecting the pressure of the inflatable bag 42. In FIG. 6, for example, the compression pressure control portion 88 performs compression with the compression pressure that is the shear stress application pressure having a pressure value P1 within the predetermined range for the predetermined time T1 before completion of the shear stress application period, i.e., the predetermined time T1 before time t1, and immediately reduces the compression pressure to the atmospheric pressure at time t1. The compression pressure control portion 88 also functions as a shear stress application control portion.

Returning to FIG. 5, the vascular stiffness measurement control portion 104 first determines the systolic blood pressure value Ps and the diastolic blood pressure value Pd of the living body from the shape of the arterial vessel 29a of the living body shown in the ultrasonic cross-sectional image generated by the ultrasonic signal processing portion 84 and the shape after the compression by the compression pressure control portion 88. Therefore, in a process of increasing the compression pressure to an increased pressure value set higher than the systolic blood pressure value Ps of the living body and then decreasing the compression pressure at a predetermined pressure reduction rate, for example, 3 to 6 mmHg/sec, the vascular stiffness measurement control portion 104 determines as the systolic blood pressure value Ps the compression pressure at the time of occurrence of the pulse wave in which the cross section of the arterial vessel 29a of the living body shown in the ultrasonic cross-sectional image is opened within one pulse wave period, and as the diastolic blood pressure value Pd the compression pressure when the cross section of the arterial vessel 29a is no longer closed within one pulse wave period, and stores a vascular diameter Ds of the arterial vessel 29a at the time of determination of the systolic blood pressure value Ps and a vascular diameter Dd of the arterial vessel 29a at the time of determination of the diastolic blood pressure value Pd together with the systolic blood pressure value Ps and the diastolic blood pressure value Pd.

Subsequently, the vascular stiffness measurement control portion 104 calculates the stiffness parameter β indicative of the stiffness of the arterial vessel 29a based on the vascular diameter Ds of the arterial vessel 29a at the time of determination of the systolic blood pressure value Ps, the vascular diameter Dd of the arterial vessel 29a at the time of determination of the diastolic blood pressure value Pd, the systolic blood pressure value Ps, and the diastolic blood pressure value Pd from the following preliminarily stored equation (stiffness parameter calculation equation) for obtaining the stiffness parameter β:

β=(ln Ps−ln Pd)/((Ds−Dd)/D0)

D0 of the stiffness parameter calculation equation should originally be the vascular diameter without pressure application; however, this diameter cannot clinically be measured, and therefore, when used as a clinical index, a vascular diameter including a vascular wall thickness (=Dd+2IMT) is used. This IMT is a thickness of a composite body of the intima and the media, for example.

Generally, a two-dimensional coordinate system between an axis representative of the vascular diameter D and an axis representative of the blood pressure P has a nonlinear relationship in which the increase in the vascular diameter D is saturated with respect to the increase in the blood pressure P; however, the relationship can be represented as a linear relationship in a semilogarithmic graph in which the axis representative of the blood pressure P in the two-dimensional coordinate system is replaced with an axis representative of a logarithmic value ln P of the blood pressure. In this linear relationship, the stiffness parameter β is an index used instead of an elastic modulus Ep in a relationship of the equation of the elastic modulus Ep (Ep=ΔP/2(ΔD/D)) established by a change rate ΔD of the vascular diameter D and a change amount ΔP of the blood pressure P when (ln Ps−ln Pd) is used instead of ΔP. The stiffness parameter calculation equation is derived from the relationship described above.

The display control portion 92 displays on the image display device 20 the diameter of the arterial vessel 29a, the endothelium diameter (lumen diameter) d1 that is the diameter of the endothelium 70, the dilation rate (change rate) R (%) of the vascular lumen diameter d1 of the arterial vessel 29a indicative of FMD (flow-mediated dilation) after ischemic reactive hyperemia, the systolic blood pressure value Ps and the diastolic blood pressure value Pd of the living body, the stiffness parameter β indicative of the stiffness of the arterial vessel 29a, etc., which are calculated in the vascular state evaluating portion 90.

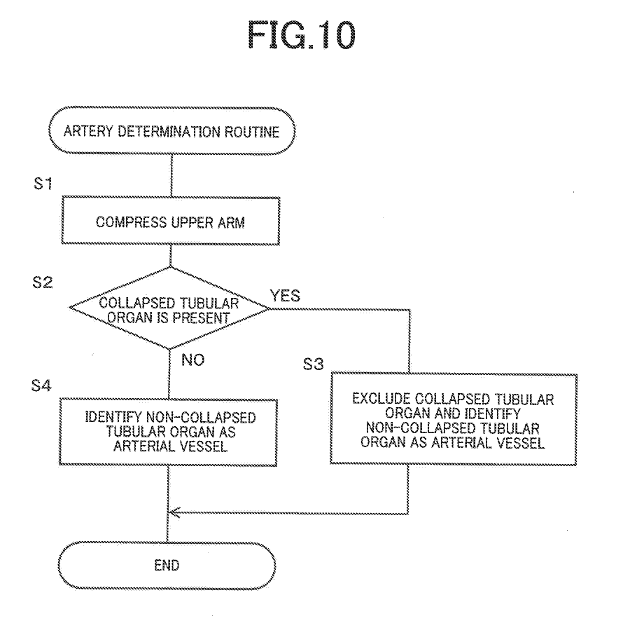

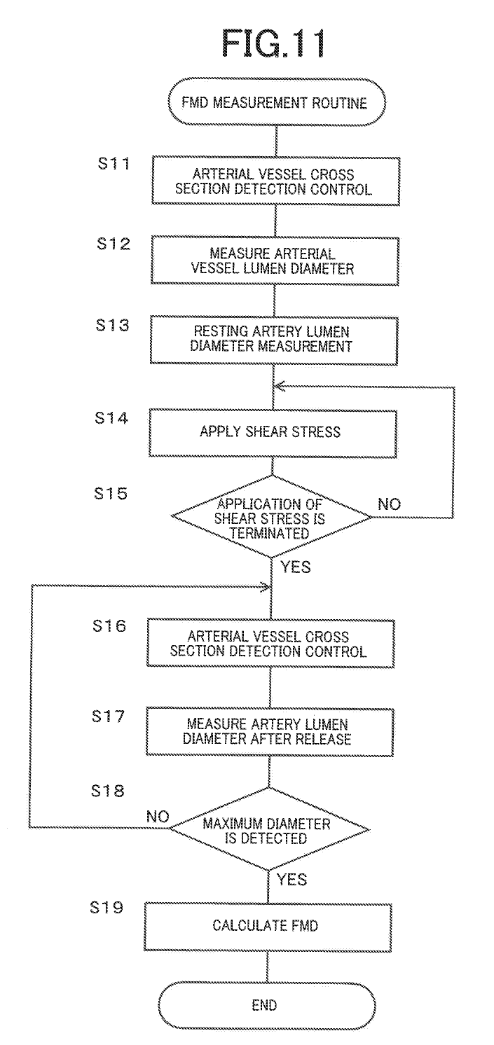

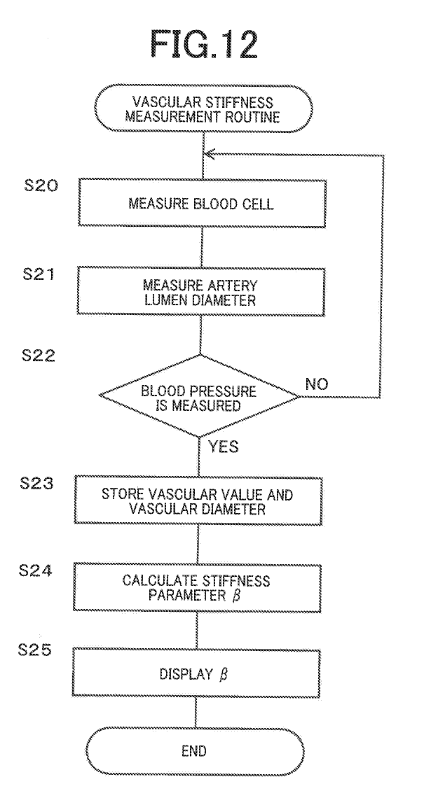

FIGS. 10, 11, and 12 are flowcharts for explaining a main portion of control operation of the electronic control device 22; FIG. 10 shows an artery determination routine corresponding to the vascular state evaluating portion 90; FIG. 11 shows an FMD measurement routine corresponding to the vascular state evaluating portion 90; and FIG. 12 shows an arterial stiffness measurement routine corresponding to the vascular state evaluating portion 90. The artery determination routine, the FMD measurement routine, and the arterial stiffness measurement routine may be executed in conjunction with an activation operation of the arterial vessel evaluating device 10 or may be executed in response to individual activation operations.

At step S1 (hereinafter, step is omitted) in the artery determination routine of FIG. 10 corresponding to the arterial vessel determining portion 100, the upper arm 29 is compressed at a pressure higher than the venous pressure and lower than the diastolic blood pressure value Pd by the compression pressure control portion 88. At S2, it is determined whether a collapsed tubular organ is present in images showing multiple tubular organs existing in an ultrasonic cross-sectional image. If the determination of S2 is affirmative, a process is executed at S3 to exclude the collapsed tubular organ and to determine and identify a non-collapsed tubular organ as the arterial vessel 29a in the ultrasonic cross-sectional image. If the determination of S2 is negative, a process is executed at S4 to determine and identify a non-collapsed tubular organ as the arterial vessel 29a in the ultrasonic cross-sectional image. The arterial vessel 29a identified in this way is measured in terms of the diameter of the arterial vessel 29a, the endothelium diameter (lumen diameter) d1 that is the diameter of the endothelium L1 of the arterial vessel 29a, the dilation rate (change rate) R (%) of the vascular lumen diameter d1 of the arterial vessel 29a indicative of FMD (flow-mediated dilation) after ischemic reactive hyperemia, the systolic blood pressure value Ps and the diastolic blood pressure value Pd of the living body, the stiffness parameter β indicative of the stiffness of the arterial vessel 29a, etc.

At S11 in the FMD measurement routine of FIG. 11 corresponding to the vascular state evaluating portion 90, a cross-sectional image of the artery vessel 29a is extracted by using a template etc. from an image identified as the artery in the ultrasonic cross-sectional image obtained by the ultrasonic signal processing portion 84.

At S12, for example, the endothelium diameter (lumen diameter) d1, i.e., the endothelium diameter of the endothelium L1, is measured as the diameter of the artery 29 from the cross-sectional image of the arterial vessel 29a extracted at S11. At S13, the endothelium diameter (lumen diameter) d1 measured at S12 is stored as the lumen diameter da at rest. Time t0 of FIG. 6 shows this state.

Subsequently, at S14, the upper arm 29 is compressed by the living body compressing device 18 to achieve the shear stress application pressure at which a shear stress can efficiently be applied to the endothelium L1 through the occurrence of turbulence of blood due to repetition of opening/closing of the arterial vessel 29a, so as to start application of the shear stress based on the blood flow to the arterial vessel 29a in the upper arm 29. Time t0 of FIG. 6 shows this state. In this application of the shear stress, the compression pressure by the living body compressing device 18 is controlled within the predetermined pressure range P1 to achieve pulsation having a section in which the arterial vessel 29a is flatly compressed (flatly closed) within one pulse wave period during the predetermined time T1 of several to several tens of beats or several to several tens of seconds, for example. The compression pressure may be controlled to be maintained at a constant value set within the predetermined pressure range P1 in the predetermined time T1 as shown in FIG. 7, for example, or may be controlled to pass through the predetermined pressure range P1 in the predetermined time T1 in an increasing process or a decreasing process at about 5 to 6 mmHg/sec, for example, as shown in FIG. 8 or 9, for example.

Subsequently, at S15, it is determined whether the predetermined time T1 has elapsed from the start of the application of the shear stress. While the determination of S15 is negative, S14 and the following steps are repeatedly executed, and when the determination of S15 becomes affirmative, the same arterial vessel cross-section detection control routine as S11 is executed at S16. As described above, a turbulent flow is repeatedly generated in the blood flow in the arterial vessel 29a that is repeatedly opened and closed, and a shear stress is repeatedly applied to the endothelium L1 of the blood vessel 29a at the measurement part. This causes production of nitric oxide (NO) from the endothelium L1 of the arterial vessel 29a occurs, resulting in a temporary increase phenomenon of the endothelium diameter d1 of the arterial vessel 29a due to relaxation of the smooth muscle dependent on nitric oxide.

In this state, at S16, the same arterial vessel cross-section detection control routine as S11 is executed for each scanning of the ultrasonic probe 12 repeated in predetermined cycles. At S17, as in S12, for example, the endothelium diameter (lumen diameter) d1, i.e., the diameter of the endothelium L1, is measured as the diameter of the arterial vessel 29a for each scanning from the cross-sectional image of the arterial vessel 29a generated in S16, and the sequentially measured endothelium diameter (lumen diameter) d1 is successively stored as the lumen diameter d1 after release of blood flow restriction. This state is shown after time t1 in FIG. 6. For the measurement of the lumen diameter d1 after release of blood flow restriction, S16 and the following steps are repeatedly executed until it is determined at S18 that the lumen diameter d of the arterial vessel 29a after the release of blood flow restriction reaches the maximum value d_{MAX} as indicated at time t3 of FIG. 6.

However, when it is determined at S18 that the lumen diameter d of the arterial vessel 29a after application of the shear stress has reached the maximum value dm x, the dilation rate (change rate) R (%) [=100*(d_{MAX}−da)/da] of the vascular lumen diameter d1 indicative of FMD (flow-mediated dilation) after ischemic reactive hyperemia for evaluating the endothelial function of the arterial vessel 29a is calculated at S19 based on the maximum value d_{MAX} determined at S18 and the endothelium diameter da that is the diameter of the endothelium L1 of the arterial vessel 29a at rest obtained at S13, and is displayed on the image display device 20 by the display control portion 92.

At S20 in the arterial stiffness measurement routine of FIG. 12 corresponding to the vascular stiffness measurement control portion 104, in the process of increasing the compression pressure to the upper arm 29 by the living body compressing device 18 to a pressure higher than the pressure at which the blood vessel of the living body is completely flatly pressed in one pulse wave period and then decreasing the compression pressure at a predetermined rate, for example, 3 to 6 mmHg/sec, the systolic blood pressure value Ps is determined as the compression pressure at the time of occurrence of the first pulse wave in which the cross section of the arterial vessel 29a of the living body shown in the ultrasonic cross-sectional image is opened within one pulse wave period, and the diastolic blood pressure value Pd is determined as the compression pressure at the time of occurrence of the pulse wave when the cross section of the arterial vessel 29a is no longer closed within one pulse wave period, before the compression pressure is released. Subsequently, at S21, the vascular diameter Ds of the arterial vessel 29a at the time of determination of the systolic blood pressure value Ps and the vascular diameter Dd of the arterial vessel 29a at the time of determination of the diastolic blood pressure value Pd are measured from the cross sectional image of the arterial vessel 29a of the living body shown in the ultrasonic cross-sectional image. Subsequently, at S22, it is determined whether the blood pressure measurement is completed. While the determination of S22 is negative, S20 and the following steps are repeatedly executed, and when the determination is affirmative, the vascular diameter Ds of the arterial vessel 29a at the time of determination of the systolic blood pressure value Ps and the vascular diameter Dd of the arterial vessel 29a at the time of determination of the diastolic blood pressure value Pd are stored together with the systolic blood pressure value Ps and the diastolic blood pressure value Pd at S23.

Subsequently, at S24, the stiffness parameter 3 corresponding to the stiffness of the arterial vessel 29a is calculated from the stiffness parameter calculation equation described above based on the vascular diameter Ds of the arterial vessel 29a at the time of determination of the systolic blood pressure value Ps and the vascular diameter Dd of the arterial vessel 29a at the time of determination of the diastolic blood pressure value Pd as well as the systolic blood pressure value Ps and the diastolic blood pressure value Pd, stored at S23 described above. At S25, the stiffness parameter β is displayed on the display device 20.

As described above, the arterial vessel evaluating device 10 of this embodiment includes the ultrasonic cross-sectional image measuring device comprising: the living body compressing device 18 having the compression band 40, which has an annular shape, wrapped around a portion of the upper arm 29 for tightening the portion of the upper arm 29, the ultrasonic transmission plate material 26 that is provided as a portion of the compression band 40 and that can be brought into close contact with the portion of the upper arm 29, and the inflatable bag (actuator) 42 capable of adjusting the tension of the compression band 40 to change the compression pressure of the plate material 26 to the upper arm 29; the closed container 16 having the opening 24 closed by the ultrasonic transmission plate material 26 and filled with the oil 28; the ultrasonic probe 14 housed in the closed container 16 and transmitting and receiving ultrasonic waves through the ultrasonic transmission plate material 26 to and from the arterial vessel 29a; and the ultrasonic signal processing portion 84 generating an ultrasonic cross-sectional image based on the ultrasonic signal received by the ultrasonic probe 14, and according to the ultrasonic cross-sectional image measuring device, the cross-sectional image in the upper arm 29 compressed by the living body compressing device 18 is accurately obtained. Specifically, since the portion of the upper arm 20 is fixed by the annular compression band 40, the influence in the cross sectional image due to body motion of a person to be measured is avoided, and the part of the upper arm 29 compressed by the ultrasonic transmission plate material 26 of the living body compressing device 18 coincides with the position of the cross-sectional image in the upper arm 29 obtained through the ultrasonic transmission plate material 26 by the ultrasonic probe 14, so that the shape of the cross-sectional image in the upper arm 29 with respect to the compression pressure by the living body compressing device 18 can accurately be obtained.

In the arterial vessel evaluating device 10 of this embodiment, the electronic control device 22 changes the compression pressure applied to a portion of the upper arm 29 by the living body compressing device 18 based on the ultrasonic cross-sectional image, so that the compression pressure can be changed such that the arterial vessel 29a in the upper arm 29 in the ultrasonic cross-sectional image has a desired shape. For example, the electronic control device 22 determines whether the arterial vessel 29a is collapsed into a flatly pressed state, i.e., a flat shape, based on the cross-sectional shape of the arterial vessel 29a, and can change the compression pressure applied to the portion of the upper arm 29 by the living body compressing device 18 such that the flatly pressed state is achieved in a portion or whole of pulse period of one beat.

In the arterial vessel evaluating device 10 of this embodiment, the electronic control device 22 controls the compression pressure applied to the upper arm 29 by the living body compressing device 18 so as to maintain the predetermined number of pulses in which the arterial vessel 29a is put into the collapsed state, for example, the flatly pressed state, in a portion of each pulse wave period of the arterial vessel 29a in the upper arm 20 based on the ultrasonic cross-sectional image when the vascular dilation of the arterial vessel 29a in the upper arm 20 is measured. As a result, a turbulent flow is repeatedly generated in synchronization with pulses in the brachial artery 29a, so that a shear stress is efficiently applied to the endothelium L1 of the brachial artery 29a. For example, as compared to the conventional FMD (flow-mediated dilation) measurement in which the shear stress is applied by releasing the brachial artery 29a after five minutes of ischemia, the shear stress is applied in a short time. Therefore, the FMD measurement can be performed in a short time.

In the arterial vessel evaluating device 10 of this embodiment, after controlling the compression pressure applied to the upper arm 29 by the living body compressing device 18 so as to maintain the predetermined number of pulses or the predetermined time in which the brachial artery 29a in the upper arm 29 is flatly pressed in each pulse wave period of the brachial artery 29a based on the ultrasonic cross-sectional image so that the shear stress is applied to the brachial artery 29a, the electronic control device 22 releases the compression applied by the living body compressing device 18 and calculates a diameter expansion ratio (the dilation rate R of the lumen diameter) of the brachial artery 29a based on the ultrasonic cross-sectional image, and therefore, the FMD (flow-mediated dilation) measurement is performed in a short time.

In the arterial vessel evaluating device 10 of this embodiment, the electronic control device 22 calculates and outputs an index indicative of the stiffness of the blood vessel of the brachial artery 29a based on the ratio between a change in the shape of the brachial artery 29a in the upper arm 29 obtained from the ultrasonic cross-sectional image and a change in the compression pressure applied by the living body compressing device 18, and therefore, diagnosis can be made based on the stiffness of the blood vessel of the brachial artery 29a. For example, diagnosis can more accurately be made for arteriosclerosis by combining the index with the diameter expansion ratio (the dilation rate R of the lumen diameter) of the artery after applying the shear stress to the brachial artery 29a.

In the arterial vessel evaluating device 10 of this embodiment, the electronic control device 22 determines whether some of multiple tubular organs in the upper arm 29 are arteries or veins based on whether arterial vessels are flatly pressed due to an increase in the compression pressure applied by the living body compressing device 18 before a puncturing operation to the arterial vessel 29a. This eliminates misidentification of the blood vessel at the time of the puncturing operation, and positions of a needle and a vein are confirmed from the ultrasonic cross-sectional image during the puncturing operation, so that the operation of puncturing the vein with the needle becomes more reliable and easier. Particularly, this is effective when the vein is a central vein.

Although the embodiment of the present invention has been described with reference to the drawings, the present invention is also applied in other forms.

For example, although the closed container 16 is sealed so as not to leak the filled oil 28 in the embodiment described above, the oil 28 may be provided such that a space is formed in the closed container 16. Instead of the closed container 16, for example, an open type container having a breather plug etc. may be used for suppressing leakage of the oil 28 and forming an air passage for equalizing an internal pressure and an external pressure.

Although the living body compressing device 18 compresses a portion of the upper arm 29 in the embodiment described above, the device 18 may compress a forearm of the living body, a lower limb such as a thigh of the living body, etc.

The ultrasonic probe 14 is an H-shaped hybrid ultrasonic probe having two rows of the first short-axis ultrasonic array probe A and the second short-axis ultrasonic array probe B parallel to each other and the long-axis ultrasonic array probe C coupling the longitudinal-direction central portions of the ultrasonic array probes A and B in a plane or may be a probe having at least one pair of ultrasonic array probes having longitudinal directions crossing each other in a plane. Although crossing angle between the pair of ultrasonic array probes is preferably a right angle, the angle may not necessarily be a right angle if somewhat complicated calculations are allowed.

Although the shape of the brachial artery 29a is measured in the arterial vessel evaluating device 10 of the embodiment, a shape of a tubular organ such as a vein or a lymph vessel may be measured.

Although the inflatable bag 42 is included as the actuator in the compression band 40 of the embodiment, an actuator such as an air cylinder and a motor may be included instead.

Although the preferred embodiment of the present invention has been described in detail with reference to the drawings, the present invention is not limited thereto and is implemented with other various modifications applied without departing from the spirit thereof.

### REFERENCE SIGNS LIST

10: Arterial vessel evaluating device (Ultrasonic cross-sectional image measuring device) 12: Base 14: Ultrasonic probe 16: Closed container (Container) 18: Living body compressing device 20: Display device 22: Electronic control device (Control device) 24: Opening 26: Ultrasonic transmission plate material 28: Oil (Liquid) 29: Upper arm (Living body) 29a: Arterial vessel 30: Upper arm rest 32: Bracket 36: Palm rest 38: Flexible belt 40: Compression band 42: Inflatable bag (Actuator) 44: Probe surface 46: Base member 48: Multi-axis positioning device 52: Positioning motor drive circuit 58: Air pump 60: Pressure control valve 64: Pressure sensor 78: Positioning motor drive control portion 80: Ultrasonic drive control portion 82: Detection processing portion 84: Ultrasonic signal processing portion 88: Compression pressure control portion 90: Vascular state evaluating portion 92: Display control portion 100: Vascular shape calculating portion 102: Vascular dilation rate measurement portion 104: Vascular stiffness measurement portion A: First short-axis ultrasonic array probe B: Second short-axis ultrasonic array probe C: Long-axis ultrasonic array probe

1. An ultrasonic cross-sectional image measuring device measuring an ultrasonic cross-sectional image in a living body corresponding to a change in compression pressure to the living body, comprising:
a living body compressing device including an annular compression band wrapped around a portion of the living body for tightening the portion of the living body, an ultrasonic transmission plate material allowing transmission of ultrasonic waves, disposed on a portion of the compression band, and brought into close contact with the portion of the living body, and an actuator adjusting a tension of the compression band to change a compression pressure of the ultrasonic transmission plate material to the living body;
a container including an opening closed by the ultrasonic transmission plate material and filled with a liquid;
an ultrasonic probe housed in the container and transmitting and receiving ultrasonic waves through the ultrasonic transmission plate material to and from the portion of the living body; and
a control device generating an ultrasonic cross-sectional image based on an ultrasonic signal received by the ultrasonic probe.

2. The ultrasonic cross-sectional image measuring device according to claim 1, wherein the control device changes the compression pressure applied to the portion of the living body by the living body compressing device based on the ultrasonic cross-sectional image.

3. The ultrasonic cross-sectional image measuring device according to claim 1, wherein
the control device controls the compression pressure applied to the portion of the living body by the living body compressing device so as to maintain a predetermined number of pulses in which an artery in the living body is put into a collapsed state in a portion of each pulse wave period of the living body based on the ultrasonic cross-sectional image when vascular dilation of the living body is measured.

4. The ultrasonic cross-sectional image measuring device according to claim 1, wherein
after controlling the compression pressure applied to the portion of the living body by the living body compressing device so as to maintain a predetermined number of pulses in which an artery in the living body is collapsed in each pulse wave period of the living body based on the ultrasonic cross-sectional image so that a shear stress is applied to the artery in the living body, the control device releases the compression applied by the living body compressing device and calculates a diameter expansion ratio of the artery based on the ultrasonic cross-section image.

5. The ultrasonic cross-sectional image measuring device according to claim 1, wherein
the control device calculates and outputs an index indicative of a stiffness of a blood vessel in the living body based on a ratio between a change in shape of the blood vessel in the living body obtained from the ultrasonic cross-sectional image and a change in the compression pressure applied by the compressing device.

6. The ultrasonic cross-sectional image measuring device according to claim 1, wherein
before puncturing a blood vessel in the living body, the control device determines whether the blood vessel is a vein based on whether the blood vessel is collapsed by increasing the compression pressure applied by the compressing device.
